(19) United States
(12) Patent Application Publication
(10) Pub. No.: US 2007/0233267 A1
(43) Pub. Date: Oct. 4, 2007

(54) Application of neural networks to prosthesis fitting and balancing in joints

(76) Inventors: Farid Amirouche, Highland Park, IL (US); Carlos G. Lopez Espina, Brooklyn, NY (US)

Correspondence Address: BRINKS HOFER GILSON & LIONE, P.O. BOX 10395, CHICAGO, IL, 60610, US

(21) Appl. No.: 11/393,443

(22) Filed: Mar. 29, 2006

(51) Int. Cl. A61F 2/38

(52) U.S. Cl. 623/20.14; 623/908

(57) ABSTRACT

The present invention provides systems and methods for prosthesis fitting in joints that employ a trained neural network to predict at least one unknown set of data, such as position and contact force. The unknown data is predicted based on at least one known sensor value that is obtained intraoperatively. The predicted neural network data is made available to a physician and aids in the determination of whether to resect additional bone, release soft tissues, and/or select sizes for prosthetic components. Advantageously, increased data may be provided to a physician without the need to acquire numerous samples from a patient, and fewer sensors may be employed.

### RELATED APPLICATIONS

This application incorporates by reference applicant's co-pending applications U.S. patent application Ser. No. ______ (Attorney Docket No. 12462/5), filed concurrently herewith, entitled “Device and Method of Spacer and Trial Design During Joint Arthoplasty,” and U.S. patent application Ser. No. ______ (Attorney Docket No. 12462/6), filed concurrently herewith, entitled “Force Monitoring System.”

### BACKGROUND

1. Technical Field

This invention relates to joint replacement, and more particularly, to improving prosthesis fitting and balancing in joints by employing neural network applications.

2. Related Art

Some medical conditions may result in the degeneration of a human joint, causing a patient to consider and ultimately undergo joint replacement surgery. The long-term success of the surgery oftentimes relies upon the skill of the surgeon and may involve a long, difficult recovery process.

The materials used in a joint replacement surgery are designed to enable the joint to move like a normal joint. Various prosthetic components may be used, including metals and/or plastic components. Several metals may be used, including stainless steel, alloys of cobalt and chrome, and titanium, while the plastic components may be constructed of a durable and wear resistant polyethylene. Plastic bone cement may be used to anchor the prosthesis into the bone, however, the prosthesis may be implanted without cement when the prosthesis and the bone are designed to fit and lock together directly.

To undergo the operation, the patient is given an anesthetic while the surgeon replaces the damaged parts of the joint. For example, in knee replacement surgery, the damaged ends of the bones (i.e., the femur and the tibia) and the cartilage are replaced with metal and plastic surfaces that are shaped to restore knee movement and function. In another example, to replace a hip joint, the damaged ball (i.e., the upper end of the femur) is replaced by a metal ball attached to a metal stem fitted into the femur, and a plastic socket is implanted into the pelvis to replace the damaged socket. Although hip and knee replacements are the most common, joint replacement can be performed on other joints, including the ankle, foot, shoulder, elbow, fingers and spine.

As with all major surgical procedures, complications may occur. Some of the most common complications include thrombophlebitis, infection, and stiffness and loosening of the prosthesis. While thrombophlebitis and infection may be treated medically, stiffness and loosening of the prosthesis may require additional surgeries. One technique utilized to reduce the likelihood of stiffness and loosening relies upon the skill of the physician to align and balance the replacement joint along with ligaments and soft tissue intraoperatively, i.e., during the joint replacement operation.

During surgery, a physician may choose to insert one or more temporary components. For example, a first component known as a “spacer block” is used to help determine whether additional bone removal is necessary or to determine the size of the “trial” component to be used. The trial component then may be inserted and used for balancing the collateral ligaments, and so forth. After the trial component is used, then a permanent component is inserted into the body. For example, during a total knee replacement procedure, a femoral or tibial spacer block and/or trial may be employed to assist with the selection of appropriate permanent femoral and/or tibial prosthetic components, e.g., referred to as a tibia insert.

While temporary components such as spacers and trials serve important purposes in gathering information prior to implantation of a permanent component, one drawback associated with temporary components is that a physician may need to “try out” different spacer or trial sizes and configurations for the purpose of finding the right size and thickness, and for balancing collateral ligaments and determining an appropriate permanent prosthetic fit, which will balance the soft tissues within the body. In particular, during the early stages of a procedure, a physician may insert and remove various spacer or trial components having different configurations and gather feedback, e.g., from the patient. Several rounds of spacer and/or trial implantation and feedback may be required before an optimal component configuration is determined. However, when relying on feedback from a sedated patient, the feedback may not be accurate since it is subjectively obtained under relatively poor conditions. Thus, after surgery, relatively fast degeneration of the permanent component may result.

Some previous techniques have relied on placing sensors that are coupled to a temporary component to collect data, e.g., representative of joint contact forces and their locations. One current limitation associated with the use of sensors is that, while objective feedback is obtained, that feedback is limited to the number of sensors that are employed and the number of physical tests that are performed.

Therefore, it would be desirable to obtain enhanced feedback during prosthesis fitting and balancing in joints without increasing the burden imposed upon the physician or the patient.

### SUMMARY

The present invention provides systems and methods for prosthesis fitting and balancing in joints that employ a trained neural network to predict at least one unknown set of data, such as position and load. The unknown data is predicted based on at least one known sensor value that is obtained intraoperatively. Advantageously, by employing the neural networking techniques of the present invention, increased data may be provided to a physician without the need to acquire numerous samples from a patient, and fewer sensors may be employed. The predicted neural network data is made available to a physician and aids in the determination of whether to resect additional bone, release soft tissues, and/or select sizes for prosthetic components.

In a first embodiment of the present invention, the system comprises at least one artificial condyle and at least one bearing surface disposed in proximity to the condyle. The bearing surface is adapted to receive at least one force imposed by the condyle. In one embodiment, the condyle may be disposed at the end of the femoral component used in a total knee arthroplasty procedure, while the bearing surface may be an exterior surface of a trial insert that is disposed adjacent to the femoral component.

The system further comprises at least one sensor within the bearing surface. The sensor is responsive to a force between the condyle and the bearing surface and is capable of providing a known measurement indicative thereof. In one embodiment disclosed herein, the sensor comprises a plurality of strain gages adapted to generate a voltage in response to the forces imposed on the bearing surface. A processor having a memory is operatively coupled to the sensor, and is capable of storing values obtained by the sensor.

The prosthesis fitting and balancing system further comprises a trained neural network operatively coupled to the processor. The neural network is used to predict at least one unknown measurement based on the known measurement obtained by the sensor. In particular, the neural network may predict contact position and load values for sets of readings that were not used during training of the neural network.

Other systems, methods, features and advantages of the invention will be, or will become, apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is a front perspective view depicting an embodiment of prosthetic components fitted within a human knee.

FIG. 2 is a top perspective view illustrating components of a trial insert that may be used in conjunction with the present invention.

FIG. 3 is a bottom perspective view of the trial insert of FIG. 2.

FIG. 4 is a block diagram depicting various components of a joint prosthesis fitting and balancing system.

FIG. 5 is a schematic showing details of a neural network that may be used in conjunction with the present invention.

FIG. 6 is a schematic illustrating the input, weighting, activation and transfer function of a node of the neural network in FIG. 5.

FIG. 7 is a block diagram showing the training phase of a neural network for use in the present invention.

FIG. 8 is a block diagram depicting the use phase of a neural network for use in the present invention.

FIG. 9 is a view of a finite element model that may be used in conjunction with the present invention.

FIG. 10 is a view of an alternative finite element model that may be used in conjunction with the present invention.

### DETAILED DESCRIPTION

The present invention is directed to systems and methods for prosthesis fitting and balancing in joints using neural network applications. It will be apparent that the neural networking techniques used in conjunction with the present invention, described hereinbelow, may be applied to a variety of medical procedures. For example, with respect to total knee arthroplasty, a force may be imposed between a trial insert and a femoral component, a trial insert and a tibial component, or between the trial insert and both femoral and tibial components. Further, the techniques of the present invention are suitable for applications including, but not limited to, joint replacement surgeries performed on the shoulder, elbow, ankle, foot, fingers and spine.

It will be appreciated that while the techniques of the present invention are generally described in the context of acquiring data using a trial insert during a knee replacement procedure, data also may be acquired and/or processed while a spacer is inserted in the joint, e.g., prior to implantation of the trial insert. Alternatively, data may be acquired and/or processed while a permanent component is housed within the patient. In the latter embodiment, the permanent component may utilize the apparatus and techniques described below to provide feedback to a physician while the permanent component is housed within the patient's body, i.e., after surgery.

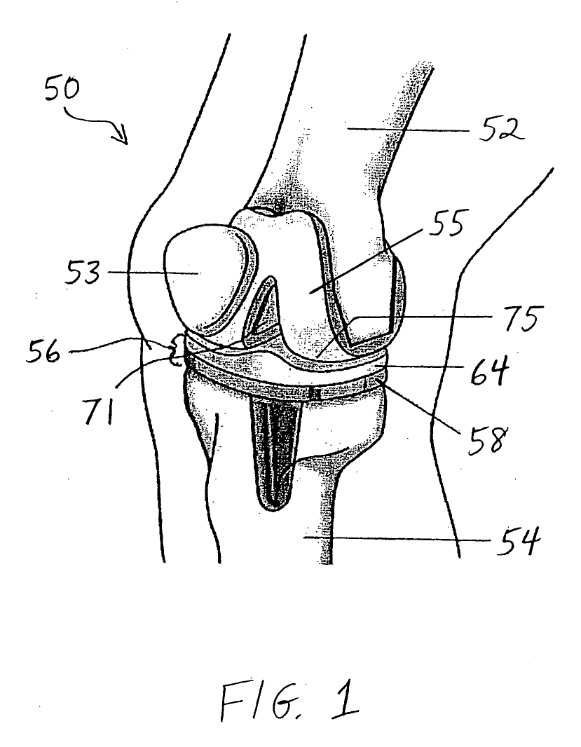

Referring now to FIG. 1, a schematic of a human knee undergoing a total knee arthroplasty (TKA) procedure is shown. In general, human knee 50 comprises femur 52, patella 53, tibia 54, a plurality of ligaments (not shown), and a plurality of muscles (not shown). An exemplary prosthesis that may be used during a TKA procedure comprises femoral component 55 and tibial component 56. Tibial component 56 may comprise tibial tray 58 and trial insert 64. Trial insert 64 may be temporarily attached to tibial tray 58, or alternatively, may be integrally formed to provide a trial bearing surface. As described in greater detail below, trial insert 64 may comprise one or more sensors that are preferably embedded and that are capable of acquiring data. The acquired data may be, for example, relating to forces and location of forces imposed upon trial insert 64 by femoral component 55.

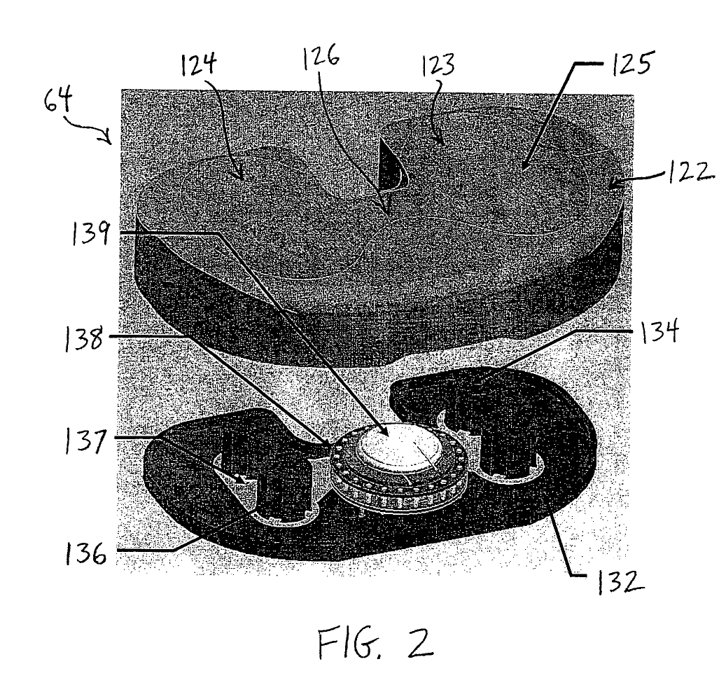

The materials used in a joint replacement surgery are designed to enable the joint to mimic the behavior or a normal knee joint. While various designs may be employed, in one embodiment, femoral component 55 may comprise a metal piece that is shaped similar to the end of a femur, i.e., having condyles 75. Condyles 75 are disposed in close proximity to a bearing surface of trial insert 64, and preferably fit closely into corresponding concave surfaces of trial insert 64, as discussed in FIG. 2 below. Femoral and tibial components 55 and 56 may comprise several metals, including stainless steel, alloys of cobalt and chrome, titanium, or another suitable material. Plastic bone cement may be used to anchor permanent prosthetic components into the femur 52 and tibia 54. Alternatively, the prosthetic components may be implanted without cement when the prosthesis and the bones are designed to fit and lock together directly, e.g., by employing a fine mesh of holes on the surface that allows the femur 52 and tibia 54 to grow into the mesh to secure the prosthetic components to the bone.

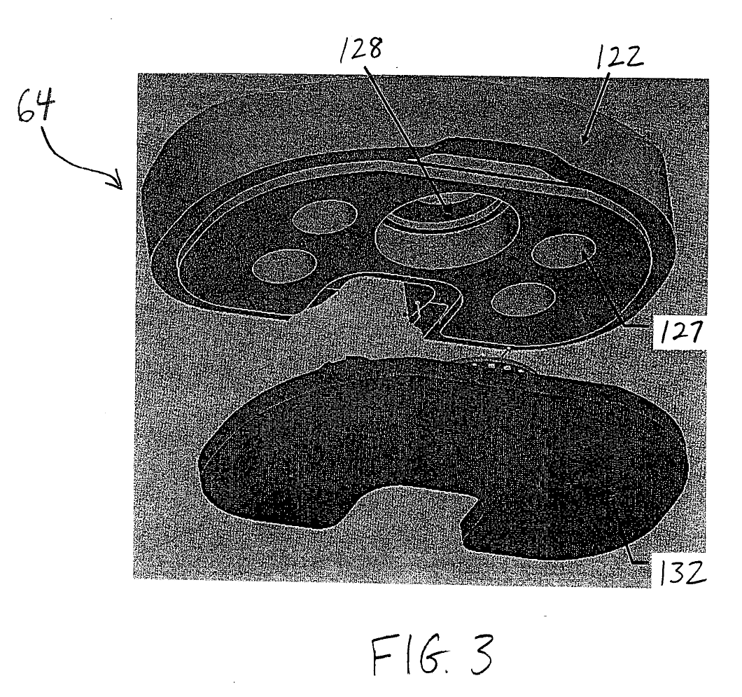

Referring now to FIGS. 2-3, exemplary trial insert 64 may be used in conjunction with the system of FIG. 1. In the described embodiment, trial insert 64 comprises first body (lower block) 132 and second body (upper block) 122. Second body 122 comprises bearing surface 123 having a pair of condyle recesses 124 and 125 formed therein. Condyle recesses 124 and 125 are shaped to closely match or otherwise accommodate condyles 75 of femoral component 55 of FIG. 1.

Second body 122 also may comprise central portion 126, which may slidably engage groove 71 in femoral component 55 (see FIG. 1). Central portion 126 may be configured to reduce or prevent lateral movement between trial insert 64 and femoral component 55, while allowing the two pieces to rotate relative to each other in a predefined range of motion similar to a biological knee, for example, between zero degrees, i.e., extension, and ninety degrees, i.e., flexion. The contact between femoral component 55 and trial insert 64 will produce deformations in the two surfaces. The deformations may be measured by at least one sensor 136, which preferably is embedded in trial insert 64, as described in greater detail below. The sensed deformation may cause an output to be generated by sensor 136.

First body 132 of trial insert 64 is adapted to be coupled to second body 122, and further is adapted to be coupled to tibial tray 58 of FIG. 1. First body 132 may be coupled to tibial tray 58 using a snap-fit connection, or other techniques that are known in the art.

In the embodiment of FIGS. 2-3, first body 132 of tibial insert 64 preferably comprises plurality of protrusions 134, such as poles, posts and/or beams. Protrusions 134 extend in a direction towards second body 122, as shown in FIG. 2. In this embodiment, sensor 136 may comprise a plurality of strain gages arranged in a pole/beam arrangement, whereby protrusions 134 both support second body 122 and transmit the load imposed to first body 132. Protrusions 134 are configured to be received within respective recesses 127 of second body 122, as shown in FIG. 3. The strain gages of sensor 136 may be coupled to their respective protrusions 134 and configured to measure compression/tension and/or bending forces imposed by femoral component 55 upon trial insert 64. Specifically, the strain gages may be adapted to generate a voltage in response to the forces imposed by condyles 75 on bearing surface 123.

It should be noted that, while one illustrative sensor embodiment having four protrusions and strain gages is depicted in FIGS. 2-3, various other sensor configurations may be employed. For example, a sensor arrangement as described in U.S. Patent Application Pub. No. 2004/0019382 A1 may be employed. Further, a person of ordinary skill in the art will readily appreciate that a single sensor, or an array of sensors, may be used to sense the deformation of trial insert 64. Furthermore, while four sensor protrusions 134 and associated strain gages are depicted in the embodiment of FIGS. 2-3, it will be apparent that greater or fewer protrusions and strain gages may be employed. In short, the important feature is that one or more sensor 136 embedded within trial insert 64 may ascertain the load and position imposed by femoral component 55 upon bearing surface 123.

First body 132 preferably further comprises printed circuit 137, data acquisition/processing unit 138, and battery 139. Printed circuit 137 is connected for communication between one or more sensors 136 and data acquisition/processing unit 138, as shown in FIG. 2. It should be noted that second body 122 comprises central recess 128 (see FIG. 3), which is configured to enclose data acquisition/processing unit 138 and battery 139. Therefore, when second body 122 is coupled to first body 132, the one or more sensors 136 are embedded within trial insert 64.

When trial insert 64 is fully assembled and disposed adjacent femoral component 55, as shown in FIG. 1, sensors 136 are responsive to the forces imposed by condyles 75 upon bearing surface 123. Furthermore, sensors 136 may provide data in a real-time, or near real-time fashion, allowing for intraoperative analysis of the data. Specifically, data acquisition/processing unit 138 may contain a memory for storing sensor data. In operation, data acquisition/processing unit 138 is adapted to receive, as an input, multiple sensor outputs created by each of the strain gages in response to the deformation of the trial insert 64. Data acquisition/processing unit 138 may be coupled to a transceiver device that is adapted to convert the multiple sensor inputs to a data stream, such as a serial data stream, and transmit the data stream, via wired or wireless connection, to processor 172 of computer 170 (see FIG. 4). The transceiver may comprise a single battery powered transceiver capable of wireless transmission, however, it may be any type of transceiver known or yet to be developed, such as a magnetically powered transceiver. The transceiver device may be embedded within first body 132, second body 122, or may be disposed external to trial insert 64.

As shown in FIG. 4, computer 170 having processor 172 and a memory coupled thereto is in communication with at least one sensor 136, which is embedded within trial insert 64. If desired, computer 170 may communicate with ancillary components 178, 180, and 182, as described in greater detail in U.S. Patent Application Pub. No. 2004/0019382 A1. For example, in one embodiment described in greater detail below, output device 180 may display neural network data in terms of a force and position of the force imposed upon a joint. Further, if desired, optional joint angle sensor 174 and optional ligament tension sensor 176 may be used during the joint replacement procedure to acquire additional data, as generally described in U.S. Patent Application Pub. No. 2004/0019382 A1.

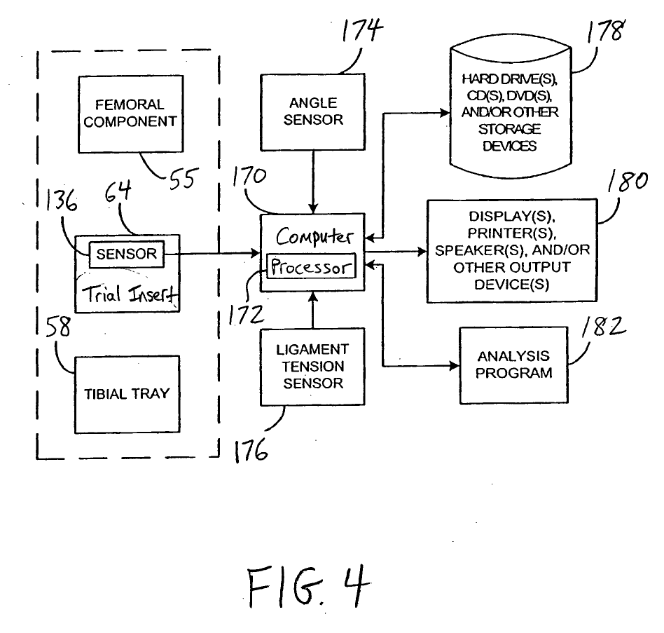

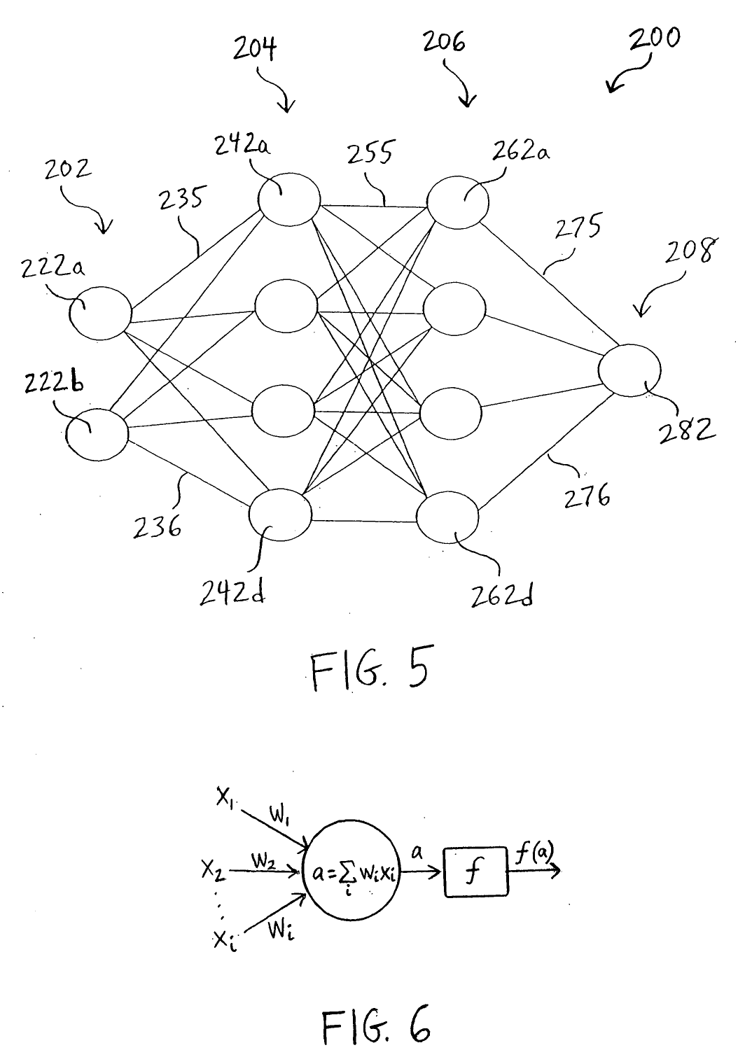

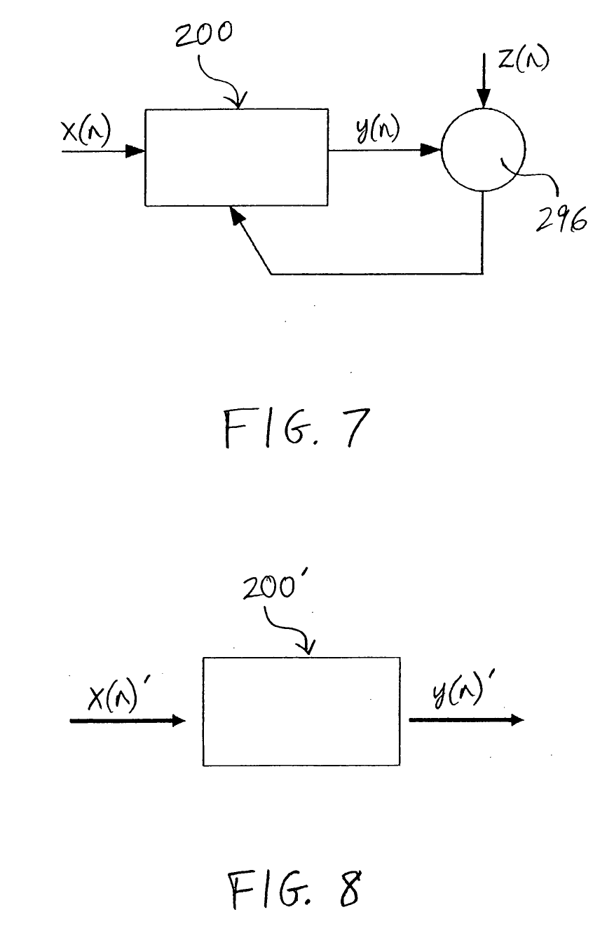

Referring now to FIGS. 5-6, an introduction to neural networking principles is provided. As will be described in greater detail below with respect to FIGS. 7-8, the neural networking principles may be used in conjunction with a joint replacement procedure to provide improved data acquisition ability and simplify the procedure. For example, known force and position data acquired by sensors 136 of trial insert 64 may be passed through a trained neural network, which can predict and output at least one previously unknown force and location in bearing surface 123 of trial insert 64. The outputted, predicted data values may be made available to a physician and used, for example, to aid in the determination of whether to resect additional bone, release soft tissues, and/or select sizes for prosthetic components during the joint replacement procedure.

In FIG. 5, a basic overview of one exemplary neural network is shown. Neural network 200 generally encompasses analytical models that are capable of predicting new variables, based on at least one known variable. The neural network comprises a specific number of “layers,” wherein each layer comprises a certain number of “neurons” or “nodes.” In the embodiment of FIG. 5, neural network 200 comprises input layer 202, first layer 204, second layer 206, and output layer 208. First and second layers 204 and 206 are commonly referred to as “hidden layers.”

In the embodiment of FIG. 5, exemplary input parameters 222a and 222b are provided. While two input parameters are shown for simplicity, it is preferred that as many input parameters as possible are included to achieve improved prediction accuracy. In the context of total joint replacement, various input parameters may be employed. The inputs may comprise “static” variables, such as the age, height, weight and other characteristics of the patient. The inputs may also comprise “dynamic” variables, such as data acquired by sensors 136 of trial insert 64. In practice, virtually any combination of static and dynamic variables may be inputted into the neural network. The aggregate input is generally represented by input layer 202.

A plurality of “connections,” which are analogous to synapses in the human brain, are employed to couple the input parameters of input layer 202 with the nodes of first layer 204. In the embodiment of FIG. 5, illustrative connection 235 couples input parameter 222a to first layer node 242a, while connection 236 couples input parameter 222b to node 242d. A different connection is employed to couple each input parameter to each node of the first layer. In FIG. 5, since there are two input parameters and four nodes in first layer 204, then eight connections total are employed between input layer 202 and first layer 204 (for simplicity, only connections 235 and 236 have been numbered). However, as noted above, any number of input parameters may be employed, and any number of first layer nodes may be selected. Therefore, the number of connections may vary widely. Moreover, as explained below, each connection has a weighted value associated therewith.

Each node in FIG. 5 is a simplified model of a neuron and transforms its input information into an output response. FIG. 6 illustrates the basic features associated with input, weighting, activation and transformation of a single node. In a first step, multiple inputs x₁-xᵢ are provided to each node. Each input x₁-xᵢ has a weighted connection w₁-wᵢ associated therewith. The activation “a” of a node is computed as the weighted sum of its inputs, as shown in FIG. 6. Finally, a transfer function “f” is applied to the activation value “a” to obtain output value “f(a)”, as shown in FIG. 6. The output value “f(a)” of a particular node then is propagated to the node of a subsequent layer for further processing.

Transfer function “f” may encompass any function whose domain comprises real numbers. While various transfer functions may be utilized, in one embodiment, a hyperbolic tangent sigmoidal function is employed for nodes within first hidden layer 204 and second hidden layer 206, and a linear transfer function is used for output layer 208. Alternatively, a step function, logistic function, and normal or Gaussian function may be employed.

In sum, any number of hidden layers may be employed between input layer 202 and output layer 208, and each hidden layer may have a variable number of nodes. Moreover, a variety of transfer functions may be used for each particular node within the neural network.

Since neural networks learn by example, many neural networks have some form of learning algorithm, whereby the weight of each connection is adjusted according to the input patterns that it is presented with. Therefore, before neural network 200 may be used to predict unknown parameters, such as contact locations and forces that may be experienced in the context of total joint replacement surgery, it is necessary to “train” neural network 200.

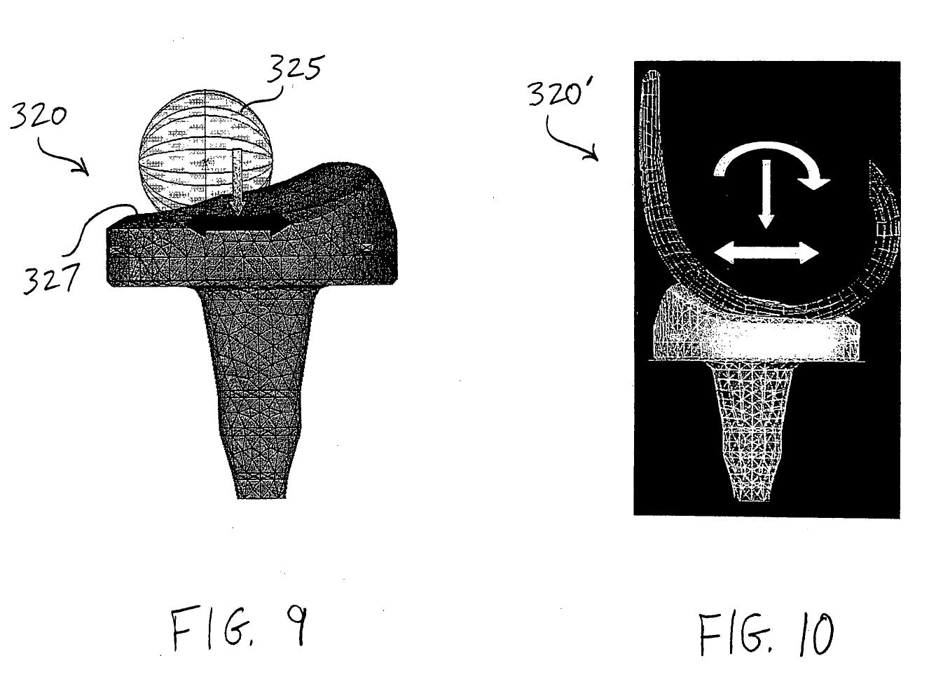

In order to effectively train neural network 200, it is important to have a substantial amount of known data stored in a database. The database may comprise information regarding known contact forces and their locations. Data samples may be acquired using various techniques. For example, as described with respect to FIGS. 9-10 below, known position and load values may be obtained using computer analysis models, such as finite element modeling. Alternatively, sample data values may be obtained using a load testing machine, such as those manufactured by Instron Corporation of Norwood, Mass. The sample data values representative of position and load may be stored in processor 172 of computer 170.

The data samples may be separated into three groups: a training set, a validation set, and a test set. The first set of known data samples may be used to train neural network 200, as described below with respect to FIG. 7. The second set of known data samples may be used for validation purposes, i.e., to implement early stop and reduce over-fitting of data, as described below. Finally, the third set of known data samples may be used to provide an error analysis on predicted sample values.

Referring now to FIG. 7, a block diagram showing the training phase of neural network 200, for use in conjunction with prosthesis fitting and balancing in joints, is described. A key feature of neural network 200 is that it may learn an input/output relationship through training. Neural network 200 may be trained using a supervised learning algorithm, as described below, to adjust the weight of the connections to reduce the error in predictions. The training data set may be used to train the neural network using MATLAB or another suitable program. In the context of a joint replacement procedure, neural network 200 may take one or more input parameters, e.g., sensor values obtained from sensor 136, and predict as output one or more unknown parameters, e.g., contact positions and loads that ultimately may be imposed upon a permanent component.

In a first training step, an input value “x(n)” is inputted into neural network 200. After being processed through neural network 200, a predicted output value, generally designated “y(n),” is obtained. It should be noted that predicted output value y(n) of FIG. 7 is the same value as output 282 of FIG. 5. Predicted output y(n) then is compared to a target value, generally designated “z(n).” Error logic 296, such as a scalar adder logic, then compares predicted output value y(n) with target value z(n).

In the context of joint replacement surgery, input value x(n) may comprise measured sensor values indicative of position and load. Further, target value z(n) may comprise known sample data representative of position and load. The known sensor values x(n) are fed through neural network 200 and predicted output y(n) is obtained. Logic 296 compares the estimated output y(n) with known target value z(n), and the weight of the connections are adjusted accordingly.

The supervised learning algorithm used to train neural network 200 may be the known Bayesian Regularization algorithm with early stopping. Alternatively, neural network 200 may learn using the Levenberg-Marquardt learning algorithm technique with early stopping, either alone or in combination with the Bayesian Regularization algorithm. Neural network 200 also may be trained using simple error back-propagation techniques, also referred to as the Widrow-Hoff learning rule.

As noted above, a set of data samples may be used for validation purposes, i.e., to implement early stop and reduce over-fitting of data. Specifically, the validation data samples may be used to determine when to stop training the neural network so that the network accurately fits data without overfitting based on noise. In general, a larger number of nodes in hidden layers 204 and 206 may produce overfitting.

Finally, a third set of known data samples may be used to provide an error analysis on predicted sample values. In other words, to verify the performance of the final model, the model is tested with the third data set to ensure that the results of the selection and training set are accurate.

Referring now to FIG. 8, a use phase of neural network 200 is shown. The use phase may be employed to predict contact forces during a joint arthroplasty procedure. Contact forces that may be experienced during or after surgery may be estimated. During surgery, only a limited number of sensors 136, e.g., four sensors, are disposed within trial insert 64. Instead of yielding data representative of four sensors, neural network 200 may use the limited data from sensors 136 to predict position and load values for numerous other locations on bearing surface 123. Advantageously, the enhanced feedback provided to the physician may be used to aid in balancing soft tissue during the arthroplasty procedure.

In FIG. 8, sensor value x(n)′ is fed through previously-trained neural network 200′ to obtain at least one previously unknown data value y(n)′. Sensor value x(n)′ may comprise data representative of load and position, as measured by sensors 136. As noted above, sensors 136 may intraoperatively collect data representative of a force imposed on bearing surface 123 during flexion or extension of the knee. During the medical procedure, the physician may maneuver the knee joint so that sensors 136 collect real-time data. This sensor data x(n)′ may be operatively coupled to processor 172, so that processor 172 may implement the trained neural network algorithms to predict unknown data values.

Advantageously, by employing neural network techniques in conjunction with data sensing techniques of the present invention, a physician may obtain significant amounts of estimated data from only a few data samples. During a prosthesis fitting procedure, the physician only needs to insert one trial insert 64 having sensors 136 embedded therein. The physician need not “try out” multiple trial inserts to determine which one is an appropriate fit before implanting a permanent component. Rather, by employing the neural networking techniques described herein, the physician may employ one trial insert 64, acquire a limited amount of force/position data, and be provided with vast amounts of data to aid in the determination of whether to resect additional bone, release soft tissues, and/or select sizes for prosthetic components during the joint replacement procedure.

Further, by employing the neural networking techniques described herein, the physician need not substantially rely on verbal feedback from a patient during a procedure. By contrast, the physician may rely on the extensive data provided by the neural network software, thereby facilitating selection of permanent prosthetic components. Moreover, it is expected that the prosthetic components will experience reduced wear post-surgery because of improved component selection and/or the ability to properly balance soft tissue during surgery based on the neural network data available to the physician.

Another advantage of using the neural network technique of the present invention in a joint replacement procedure is that the database of stored values can grow over time. For example, even after a neural network is trained and used in procedures to predict values, sensed data may be inputted and stored in the database. As the database grows, it is expected that improved data estimations will be achieved.

As noted above, it will be appreciated that while the techniques of the present invention have been described in the context of acquiring data using a trial insert during a knee replacement procedure, data also may be acquired and/or processed while a spacer is inserted in the joint, e.g., prior to implantation of the trial insert. Alternatively, data may be acquired and/or processed while a permanent component is housed within the patient. In the latter embodiment, the permanent component may utilize the apparatus and techniques described above to provide feedback to a physician while the component is housed within the patient's body, i.e., after surgery.

Referring now to FIGS. 9-10, methods for collecting data for use in creating a database of known solutions for training a neural network are provided. As noted above, in order to effectively train neural network 200, it is important to have a substantial amount of existing, known data stored in a database. In FIGS. 9-10, data samples indicative of position and load are obtained using finite element modeling. In FIG. 9, finite element model 320 is shown. A load, represented by sphere 325, is dragged over simulated bearing surface 327. The load preferably is cycled throughout bearing surface 327 in an anterior/posterior direction and a medial/lateral direction. The load imposed may range, for example, from about 0 to 400 N. Preferably, hundreds or thousands of sample data points are collected. At each load point, a sensor reading indicative of position and load is stored in the database of known solutions, e.g., in processor 172 of computer 170.

In FIG. 10, finite element model 320′ is similar to finite element model 320, with the main exception that joint flexion between 0-90 degrees is simulated. Optionally, internal rotation of the joint, e.g., between −10 to 10 degrees, may be simulated. For each simulated flexion and/or rotation condition, model 320′ imposes a load on the bearing surface to obtain numerous sample data points. The sample data is stored in the database of known solutions in processor 172 and may be used to train, validate and test neural network 200, as described above. The finite element data gathered from models 320 and 320′ may be used alone or in combination with sample data obtained using a load testing machine, such as those manufactured by Instron Corporation, as described above.

In alternative embodiments of the present invention, the outputs from sensors 136 may be transmitted to processor 172, wherein they may be captured by an analysis program 182, as shown in FIG. 4. Analysis program 182 may be a finite element analysis (“FEA”) program, such as the ANSYS Finite Element Analysis software program marketed by ANSYS Inc., located in Canonsburg, Pa., and commercially available. The FEA analysis program may display the data in a variety of formats on display 180. In one embodiment, sensor measurements captured by the FEA analysis program are displayed as both a pressure distribution graph and as a pressure topography graph, as described in U.S. Patent Application Pub. No. 2004/0019382 A1.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

What is claimed is:

1. A system for prosthesis fitting in joints, the system comprising:
at least one an artificial condyle;
at least one bearing surface disposed in proximity to the condyle, the bearing surface adapted to receive at least one force imposed by the condyle;
at least one sensor responsive to a force between the condyle and the bearing surface to provide a known measurement indicative thereof;
a processor having a memory, the processor being operatively coupled to the sensor; and
a trained neural network operatively coupled to the processor, wherein the neural network predicts at least one unknown measurement based on the known measurement.

2. The system of claim 1 wherein the bearing surface is an exterior surface of a trial insert.

3. The system of claim 2 wherein the trial insert comprises a first assembly adapted to be coupled to a second assembly, wherein the bearing surface is formed in the first assembly and the sensor is disposed within the second assembly.

4. The system of claim 1 wherein the sensor comprises a strain gage that generates a voltage in response to the forces imposed on the bearing surface.

5. The system of claim 1 wherein the unknown measurement comprises data indicative of a force imposed at a location of the bearing surface.

6. The system of claim 1 wherein the known measurement comprises data acquired by the sensor during a surgical procedure, the known measurement data being indicative of a force imposed at a location of the bearing surface.

7. The system of claim 1 further comprising a database coupled to the processor, wherein the database comprises sample data used to train the neural network.

8. The system of claim 7 wherein the database comprises data samples obtained from a finite element computer model.

9. The system of claim 7 wherein the database comprises data sample information obtained from a load testing machine.

10. The system of claim 1 wherein the sensor is embedded within the bearing surface.

11. The system of claim 1 wherein the processor is disposed external to the bearing surface.

12. A method for prosthesis fitting in joints, the method comprising:
providing an artificial condyle;
providing at least one bearing surface disposed in proximity to the condyle, the bearing surface adapted to receive at least one force imposed by the condyle;
sensing a force between the condyle and the bearing surface and providing a known measurement indicative thereof;
storing the known measurement data in a processor, the processor being operatively coupled to the sensor; and
using a trained neural network operatively coupled to the processor to predict at least one unknown measurement based on the known measurement.

13. The method of claim 12 further comprising providing sample data and training the neural network using the sample data.

14. The method of claim 13 further comprising using a finite element computer model to obtain data sample information and storing the information in the database.

15. The method of claim 14 further comprising using a load testing machine to obtain data sample information.

16. The method of claim 12 wherein the unknown measurement comprises data indicative of a force imposed at a location of the bearing surface.

17. The method of claim 12 wherein the sensor comprises a strain gage that generates a voltage in response to the force imposed by the condyle on the bearing surface.

18. A system for prosthesis fitting in joints, the system comprising:
an artificial condyle;
a trial insert having at least one bearing surface disposed in proximity to the condyle, the bearing surface adapted to receive at least one force imposed by the condyle;
at least one sensor responsive to a force between the condyle and the bearing surface and capable of providing a known measurement indicative thereof;
a processor having a memory, the processor being operatively coupled to the sensor; and
a neural network operatively coupled to the processor, wherein the neural network is used to predict at least one unknown measurement based on the known measurement.

19. The system of claim 18 wherein the trial insert comprises a first assembly adapted to be coupled to a second assembly, wherein the bearing surface is formed in the first assembly and the sensor is disposed within the second assembly.

20. The system of claim 18 wherein the sensor comprises a strain gage adapted to generate a voltage in response to a force imposed on the bearing surface.

21. The system of claim 18 wherein the unknown measurement comprises data indicative of force imposed at a location of the bearing surface.

22. The system of claim 18 wherein the known measurement comprises data acquired by the sensor during a surgical procedure, the known measurement data being indicative of a force imposed at a location of the bearing surface.

23. The system of claim 18 further comprising a database coupled to the processor, wherein the database comprises sample data used to train the neural network.

24. The system of claim 23 wherein the database comprises data samples obtained from a finite element computer model.

25. The system of claim 23 wherein the database comprises data sample information obtained from a load testing machine.

26. The system of claim 18 wherein the sensor is embedded within the bearing surface of the trial insert.
